(19) United States
(12) Patent Application Publication
(10) Pub. No.: US 2019/0269338 A1
(43) Pub. Date: Sep. 5, 2019

(54) NON-INVASIVE BLOOD PRESSURE SENSOR

(71) Applicant: Basil Leaf Technologies, LLC, Paoli, PA (US)

(72) Inventors: Basil M. Harris, Paoli, PA (US); George C. Harris, Ramsey, NJ (US); Edward L. Hepler, Malvern, PA (US); Constantine F. Harris, Wyomissing, PA (US)

(73) Assignee: Basil Leaf Technologies, LLC, Paoli, PA (US)

(21) Appl. No.: 16/347,089

(22) PCT Filed: Nov. 3, 2017
(86) PCT No.: PCT/US17/59883

§ 371 Date: May 2, 2019

Related U.S. Application Data
(60) Provisional application No. 62/417,231, filed on Nov. 3, 2016.
(60) Provisional application No. 62/432,171, filed on Dec. 9, 2016.

(51) Int. Cl. A61B 5/021; A61B 5/00; A61B 7/02

(52) U.S. Cl. CPC A61B 5/02125; A61B 7/02; A61B 5/02141; A61B 5/6806

(57) ABSTRACT

A non-invasive blood pressure sensor comprises tissue-matable sensor bodies that include a first light-source-and-photodetector pair disposed on one of the sensor bodies in a pre-determined spatial relationship for a proximal anatomical location, and a second light-source-and-photodetector pair disposed on one of the sensor bodies in a pre-determined spatial relationship for a distal anatomical location. The sensor bodies may be mounted on a support structure acting as a jig for aligning and/or spacing them. A controller receives signals from the photodetectors and calculates blood pressure by identifying peaks and valleys in time series data obtained from the photodetectors, and calculating the subject's blood pressure based on differences in time between: (i) a proximal peak detected by the first light-source-and-photodetector pair and a distal peak detected by the second light-source-and-photodetector pair; and (ii) a proximal peak detected by the first light-source-and-photodetector pair and a distal valley detected by the second light-source-and-photodetector pair.

### CROSS-REFERENCE TO RELATED APPLICATIONS

This application claims the benefit of priority of U.S. Provisional Patent Application Nos. 62/417,231, filed Nov. 3, 2016, and 62/432,171, filed Dec. 9, 2016, the entire disclosures of both of which are hereby incorporated herein by reference.

### FIELD OF THE INVENTION

The present invention relates generally to and more particularly to blood pressure measuring devices, and more particularly, to sensors and methods for automated measuring blood pressure in the body without the need for a conventional inflatable rubber cuff of a conventional sphygmomanometer, a stethoscope, or a healthcare professional skilled in the use of same.

### BACKGROUND OF THE INVENTION

Although various devices exist for measuring blood pressure, no single device can reliably measure blood pressure in a small form factor and without the need for user training.

### SUMMARY

The present invention provides a non-invasive blood pressure sensor comprising: one or more sensor bodies configured to mate with a tissue surface; a first light-source-and-photodetector pair disposed on one of the one or more sensor bodies in a pre-determined spatial relationship over a proximal anatomical location on a subject; and a second light-source-and-photodetector pair disposed on one of the one or more sensor bodies in a pre-determined spatial relationship over a distal anatomical location on the subject. The first light-source-and-photodetector pair and the second light-source-and-photodetector pair may be disposed on the one or more sensor bodies in a pre-determined spatial relationship with respect to each other. The sensor bodies may be mounted on a rigid support structure, or a flexible support structure such as a strap, a glove, a cuff, and a sleeve, each of which is configured to register with a corresponding portion of human anatomy in a predetermined fashion, to support the light-source-and-photodetector pairs in a pre-determined spatial relationship with respect to the corresponding portion of human anatomy, such that the support structure acts as a jig for aligning the sensor bodies with the human anatomy, and/or spacing the sensor bodies along the anatomy, in a predefined fashion.

The sensor's light sources may emit light having a color selected from the group consisting of ultraviolet, violet, blue, green, yellow, orange, red, near infrared, and infrared. The sensor may include a controller programmed to: receive one or more signals from the photodetectors; and calculate blood pressure values as function of at least the one or more signals received from the photodetectors after emission by the light sources. The controller may be further programmed to: identify a plurality of peaks and valleys over a time series of data obtained from the photodetectors; and calculate the subject's blood pressure based on differences in time between: (i) a proximal peak detected by the first light-source-and-photodetector pair and a distal peak detected by the second light-source-and-photodetector pair; and (ii) a proximal peak detected by the first light-source-and-photodetector pair and a distal valley detected by the second light-source-and-photodetector pair.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A depicts a non-invasive blood pressure sensor according to an embodiment of the invention.

FIGS. 1B and 1C depict an exemplary positioning of light sources and photodetectors along a subject's finger for measurement of reflectance/transflectance and transmission, respectively, according to embodiments of the invention.

FIGS. 1D and 1E depict an exemplary light source and photodetector assembly according to an embodiment of the invention.

FIG. 2 depicts the association of photodetector signals with a previously or concurrently applied color according to an embodiment of the invention.

FIG. 3 illustrates a method for controlling a non-invasive blood pressure sensor according to an embodiment of the invention.

FIG. 4A depicts a non-invasive blood glucose sensor according to an embodiment of the invention.

FIG. 4B-4J depicts portions of the blood glucose sensor of FIG. 4A.

FIGS. 4K-4L illustrate exemplary embodiments of support structures designed to register with specific portions of human anatomy according to an embodiment of the invention.

FIGS. 5A-5C depict the location of proximal and distal peaks valleys and the calculation of differences between the same according to embodiments of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

Aspects of the invention provide non-invasive blood pressure sensors. Without being bound by theory, Applicant believes that optical absorption of blood within a vessel varies in a pulsatile manner such that the application of multiple wavelengths of light will yield different transmission, reflectance, and/or transflectance spectra depending on the content of the subject's blood and that the time between these measured pulses varies based on the subject's blood pressure.

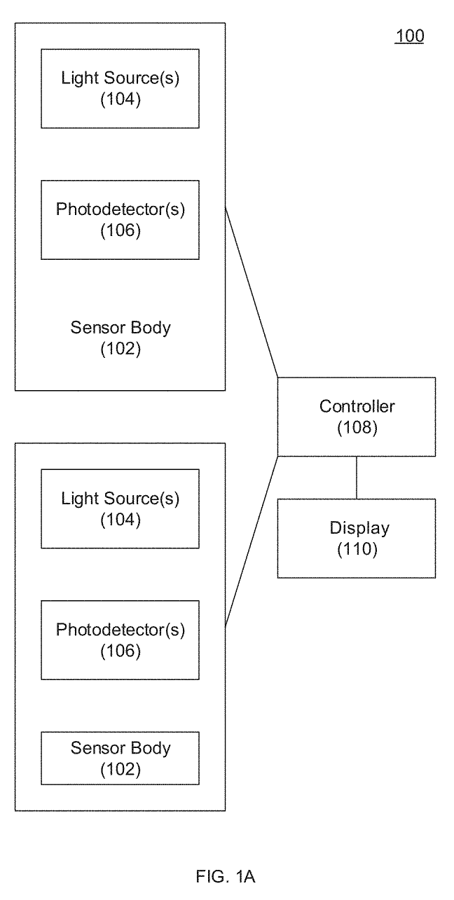

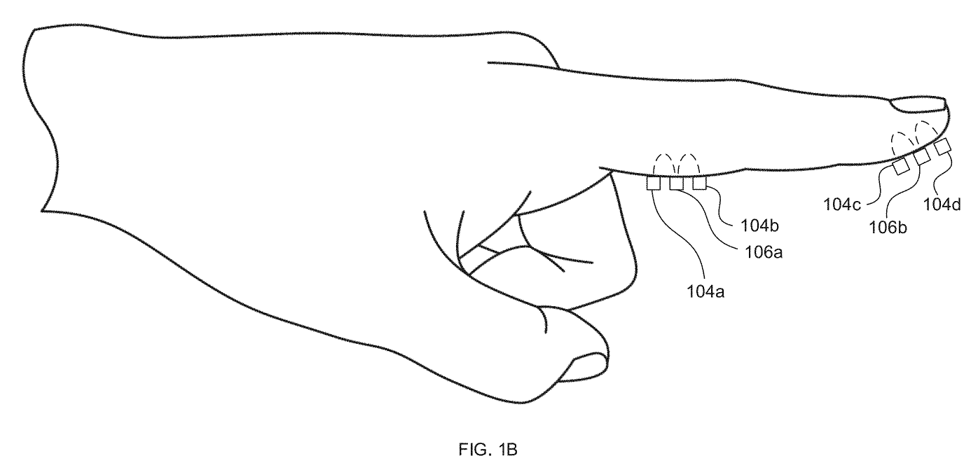

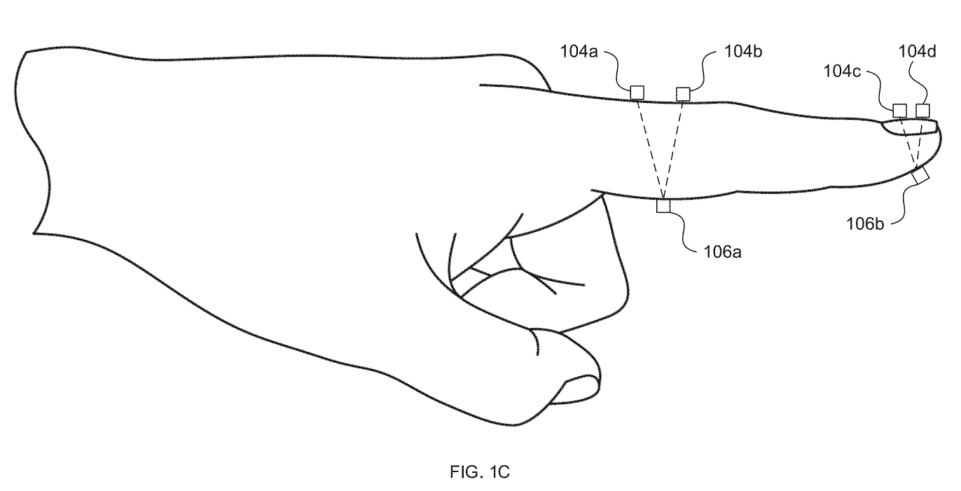

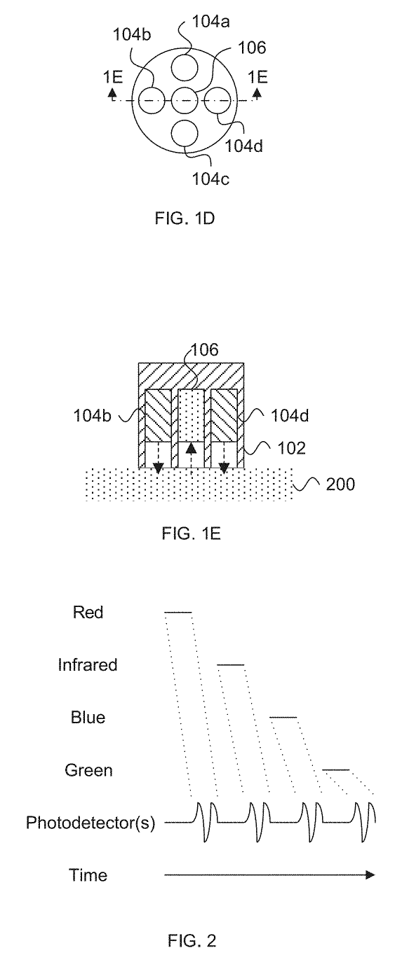

Referring to FIG. 1A, one embodiment of the invention provides a non-invasive blood pressure sensor 100 including a sensor body 102, one or more light sources 104, and one or more photodetectors 106.

In one embodiment of the invention, a single sensor body 102 can include two light-source-and-photodetector pairs that can be spaced from one another when positioned on the patient's body, in order to detect a time between optical fluctuations along a blood vessel. In other embodiments, sensor 100 can include two sensor bodies 102, each containing one or more light sources 104, and one or more photodetectors 106. The sensor bodies 102 can be positioned at positions spaced along a length of blood vessel.

Although a single frequency of any color of light (e.g., ultraviolet, violet, blue, green, yellow, orange, red, near infrared, and infrared) is believed to be sufficient to detect pulsatile variations, other embodiments can add additional light sources 104 (e.g., blue, green, red, and/or infrared light sources), which can enable detection of other values of interest.

A first light-source-and-photodetector pair can be located at a first anatomical location (e.g., the base or over a proximal phalanx of a finger) while a second light-source-and-photodetector pair can be located a second anatomical location (e.g., over a tip of the same finger), such that the first and second locations are sufficiently longitudinally spaced along the patient's anatomy that the corresponding length of the blood vessel between the respect pairs permits accurate monitoring for the purposes described herein. By way of example, a distance between sensors of at least 2.0 cm and approximately 4.0 cm to approximately 10.0 cm has been found suitable for this purpose. However, these distances are not critical and shorter distances including distances shorter than 1.0 cm are feasible, provided that corresponding hardware and signal processing speed is adequate. Positioning the first light-source-and-photodetector pair and the light-source-and-photodetector pair on the same finger is particularly advantageous because blood vessels within the human finger have a substantially constant cross-sectional dimension, which enables simplified and reliable calculations.

The light-source-and-photodetector pairs can be configured for proximal/upstream and distal/downstream application (e.g., by labeling) or can be agnostic as to positioning, in which the location of the light-source-and-photodetector pair can be specified or the controller 108 can determine their relative location based on detected signals.

The light-source-and-photodetector pairs can be placed in various configurations such that first light-source-and-photodetector pair is more proximal to the heart and second light-source-and-photodetector pair is somewhere downstream distally along the arterial system.

Examples could include placing first light-source-and-photodetector pair on a proximal portion of an extremity and a second light-source-and-photodetector pair more distal (e.g., upper arm to forearm, axilla to elbow, forearm to wrist, wrist to finger, thigh to toe, leg to ankle, and the like).

In another example, the proximal light-source-and-photodetector pair could be placed on the chest or neck and the distal light-source-and-photodetector pair could be placed on the ear, nose, or forehead.

Increased physical separation of the proximal to distal light-source-and-photodetector pairs affords a benefit in increased transit time, which is technically easier to measure; however the tradeoff is an increase in the number of variables as the pulse wave reverberates across more types of arterial blood vessels. Variations in vessel compliance and anatomic dimensions need to be considered and accounted for in those configurations.

Another way to obtain a measure of pulse transit time is to measure the time difference from the ventricular contraction on an ECG rhythm to appearance of a pulse on a distal sensor (e.g., oxygen saturation sensor on the fingertip or earlobe). This has the same tradeoff of the widely spaced proximal to distal sensors described above.

### Light Sources

Light sources 104 can be light-emitting diodes (LEDs), fiber optics, or any other device capable of generating and/or transmitting a desired wavelength to a tissue (e.g., skin) surface. Suitable LEDs are available from a variety of manufacturers and are detailed in Table 4 in the Appendix to this application.

Exemplary wavelength ranges and peak wavelengths are provided in Table 1 below.

TABLE 1
Exemplary Light Source Wavelengths

| | | Exemplary | Exemplary Peak | Exemplary Peak |
|---|---|---|---|---|
| Abbreviation | Color | Wavelength Range | Wavelength | Wavelength Range |
| B | Blue | 380-495 nm | 465 nm | 454-476 nm |
| G | Green | 495-590 nm | 515 nm | 497-533 nm |
| R | Red | 590-750 nm | 660 nm | 650-670 nm |
| IR | Infrared | 750-1000 nm | 940 nm | 915-965 nm |

In one embodiment, one or more fiber optics function as the one or more light sources by multiplexing and/or transmitting light from at least one LED or other light source located remote from the tissue surface.

### Photodetectors

Photodetector(s) 106 can be a photodiode such as a silicon photodiode (e.g., Product No. PDB-C171SM available from Luna Optoelectronics of Roanoke, Va.), a phototransistor, and the like.

Photodetector(s) 106 detect a light after partial absorption of light emitted by one of the light sources 104. For example, at least a portion of the emitted light may be absorbed by various components of blood within tissue of the subject such that the amplitude of the detected light is less than from the amplitude of the emitted light.

### Positioning of Light Sources and Photodetectors

In view of the prevalence of capillaries carrying blood skin or tissue surfaces, embodiments of the invention can be applied to most, if not all, tissue surfaces of a body without the need to position the meter or sensors over a particular blood vessel. However, particular embodiments can be configured for application to particular regions such as a finger, toe, forehead, head, ear, earlobe, chest, wrist, ankle, nostril, and the like.

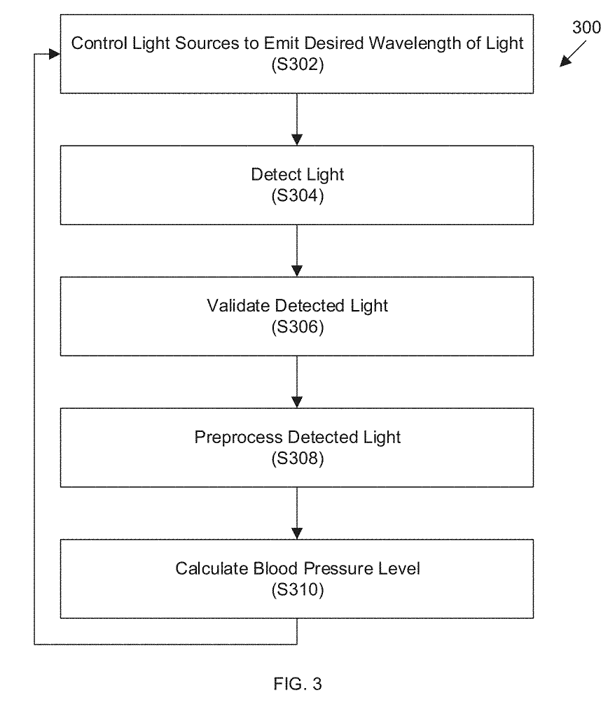

The light source(s) 104 and the photodetector(s) 106 can be positioned along the tissue surface so that the photodetector(s) 106 detect light emitted by one or more light sources 104, after absorption of some of the emitted light by blood within the tissue. As illustrated in U.S. Pat. Nos. 6,763,256, 8,818,476, and 9,314,197, photodetector(s) 106 can be located on the same surface as the light sources 104 to detect reflectance and/or transflectance of emitted light through the tissue (as also depicted in FIG. 1B) and/or the opposite side (e.g., perpendicularly opposite) of the tissue (e.g., finger) to detect transmission of the light through the tissue (as also depicted in FIG. 1C). In reflectance oximetry, the light sources are typically placed around a central photodetector (on a single body for abutting a tissue surface), which can be surrounded by a light shield to minimize detection of light that has not traveled through the subject's tissue as depicted in FIGS. 1D and 1E. Such an embodiment having an approximately 8 mm diameter is depicted in FIG. 3.11 of John TB Moyle, Pulse Oximetry 31 (2d ed. 2002).

### Sensor Housings

Referring still to FIGS. 1D and 1E, the sensor body 102 can be a wand or probe that can be placed or held over a desired tissue surface.

This assembly can be further mounted to, coupled to, and/or incorporated within a support structure component for securing the assembly against a tissue surface. Exemplary components include a strap adapted to wrap around a body part (e.g., an about 6 cm to about 10 cm strap to accommodate placement over a finger, an about 15 cm to about 23 cm strap to accommodate placement around a wrist, and the like) that can be secured to itself after wrapping around a tissue, a sleeve, a glove, and the like. The strap, sleeve, glove, cuff, spring-loaded case or clip, or other component can include one or more elastic members, hook-and-loop fasteners (e.g., those available under the VELCRO® trademark from Velcro Industries B.V. of the Netherlands Antilles), and the like.

In each case, the sensor body 102 can be designed to abut and/or register or mate with the intended anatomical structure and further support the light source(s) 104 and photodetector(s) 106 in a defined spatial relationship so that they will be properly positioned during use, according to the reflectance, transmittance, or transflectance mode of operation for which the sensor 100 is designed.

Sensor body 102 can be configured for application to one or more specific tissue surfaces. For example, sensor body 102 can be configured for application to a subject's finger and/or fingertip such as depicted in FIGS. 1B and 1C disclosed in U.S. Pat. Nos. 4,825,879, 8,554,297, 8,818,476, and 9,314,197 and U.S. Patent Application Publication Nos. 2006/0224058 and 2007/0244377, on a wrist as disclosed in U.S. Pat. No. 9,314,197, in a contact lens as disclosed in U.S. Pat. No. 8,971,978, on a heel (e.g., an infant's heel), and the like.

In various embodiment, the sensor body 102 is configured to abut and seal against the tissue surface to shield or substantially shield the light source(s) 104, the photodetector 106, and/or the tissue from ambient light. For example, in FIGS. 1D and 1E, a shell 102 surrounds light sources 104 and/or photodetector 106 such that light is directed (and sometimes collimated) toward tissue 200 and/or such that photodetector 106 can only receive light that emanates from the tissue 200. While four light sources and a single photodetector are shown in FIGS. 1D and 1E, in other embodiments, more or less light sources 104 and/or photodetectors 106 can be implemented. For other, e.g., transmission, implementations, the light sources 104 and photodetector(s) 106 can be spaced on opposite sides of tissue 200 as discussed herein, for example, in a spaced linear array along a flexible wrap.

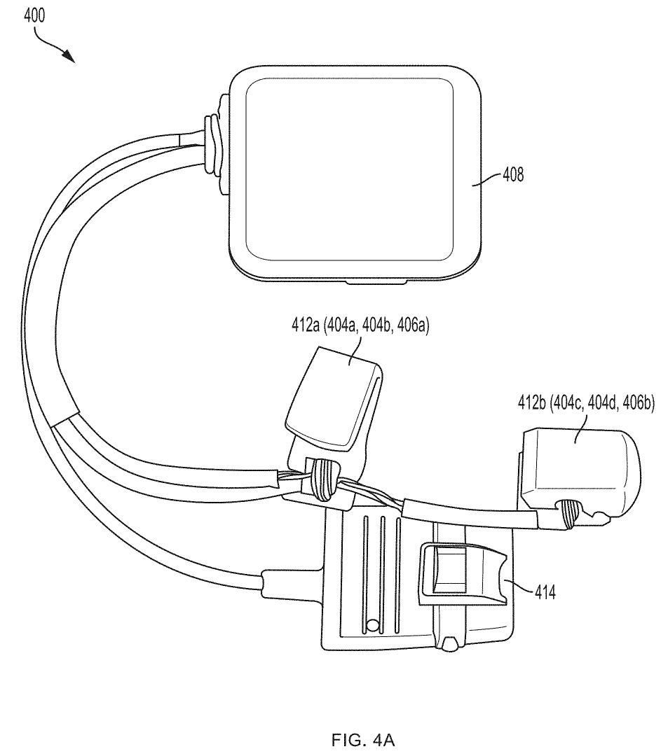

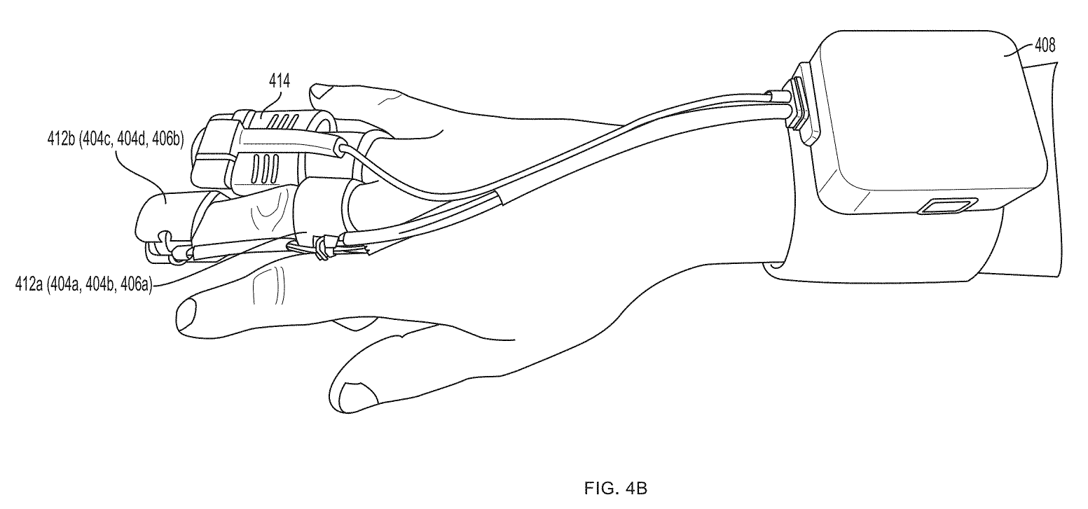

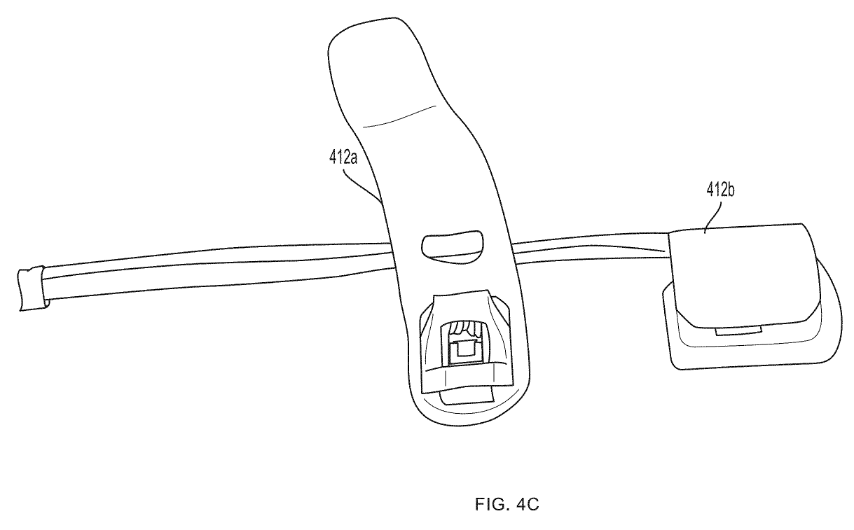

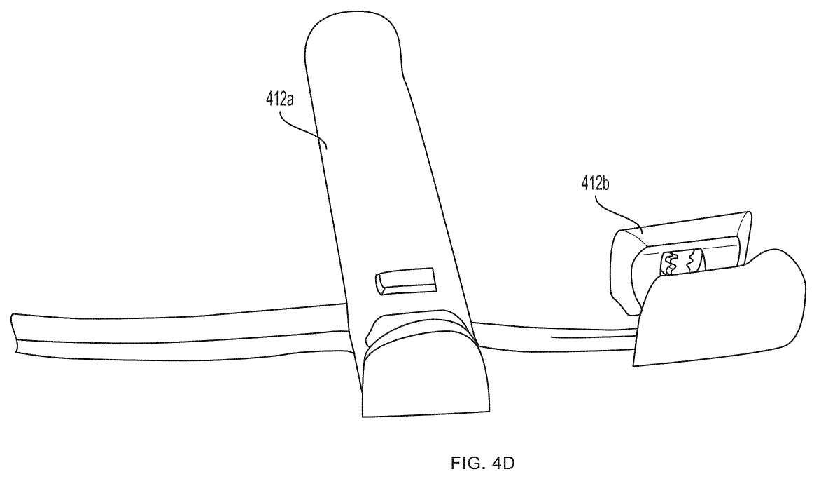

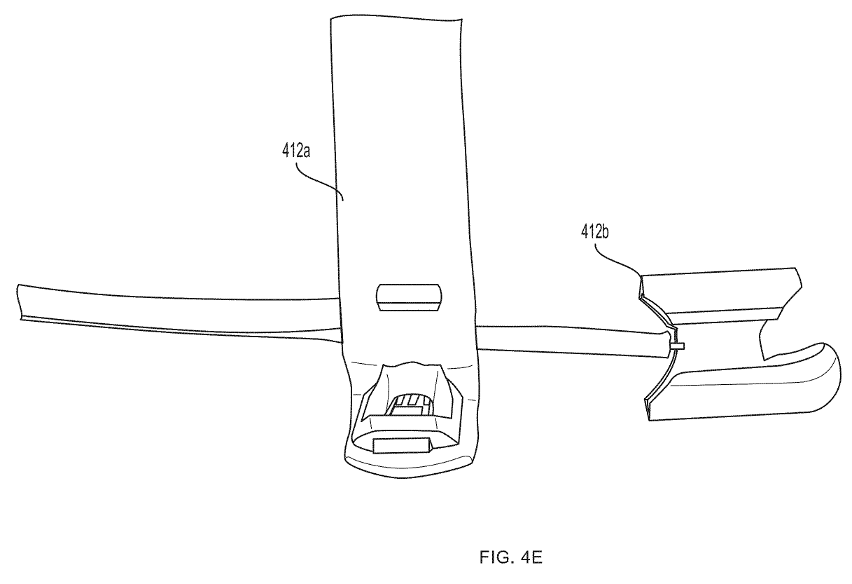

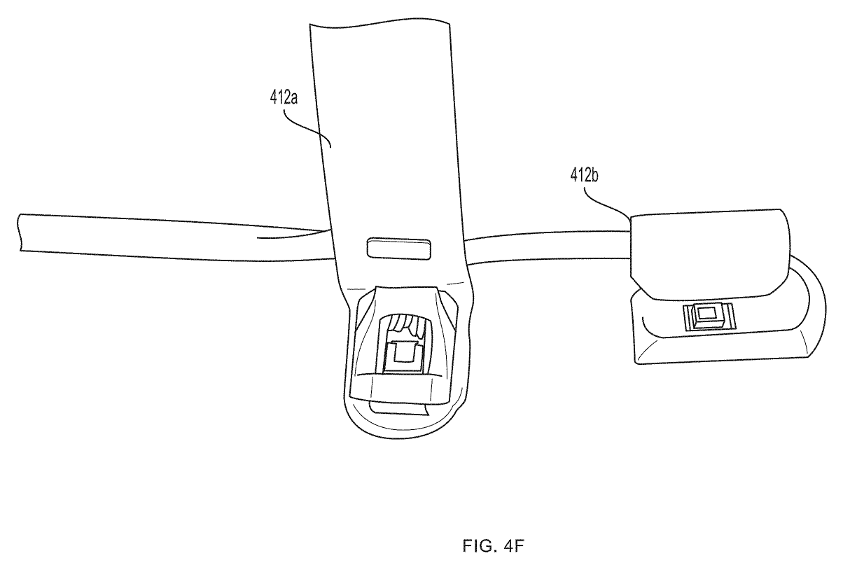

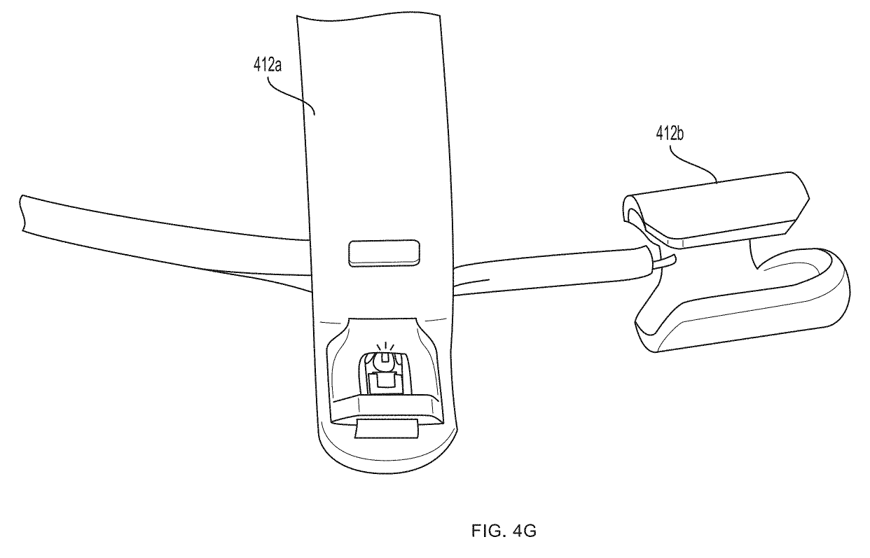

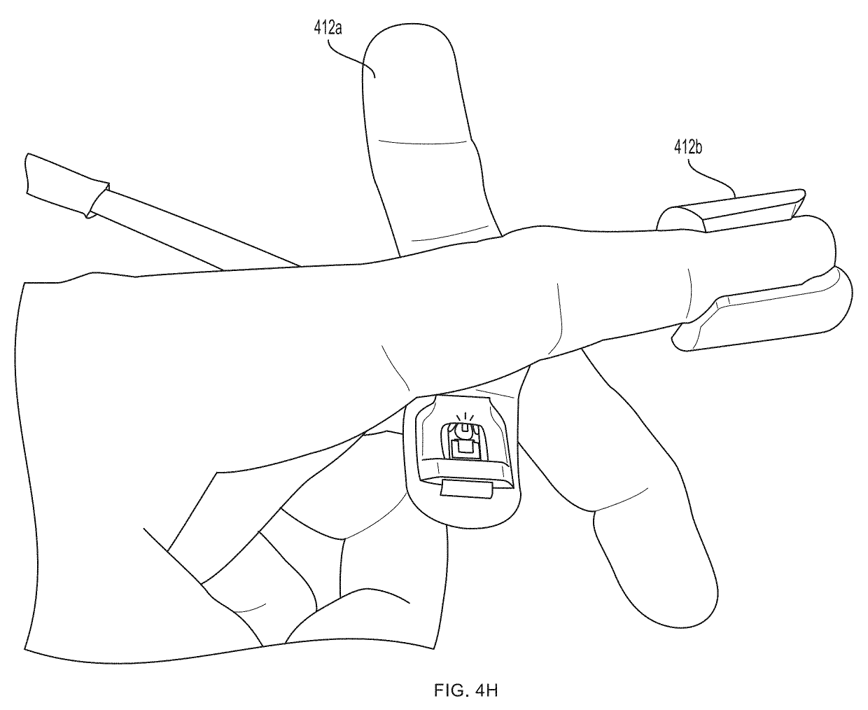

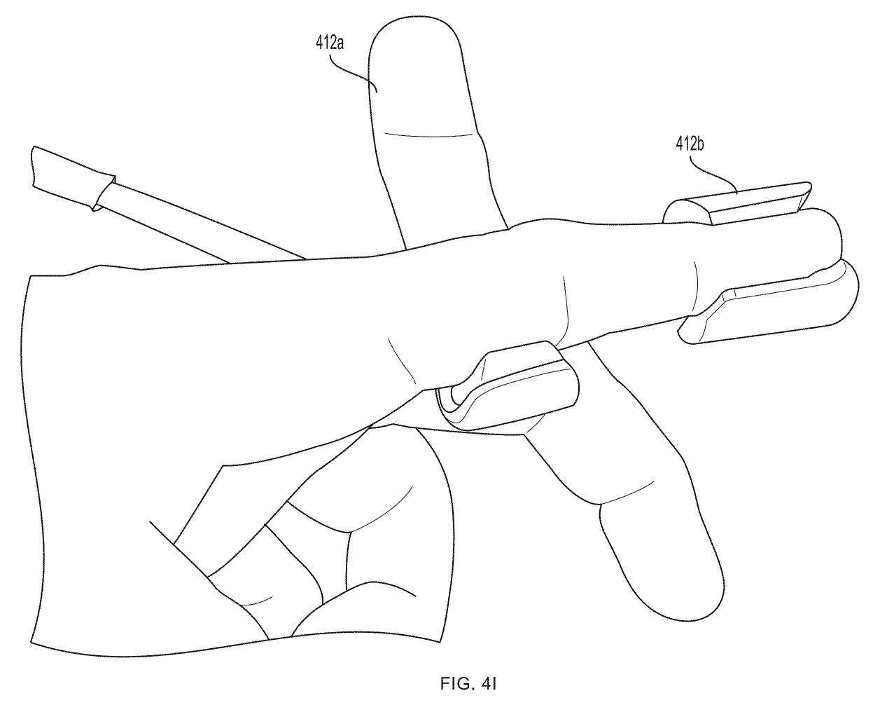

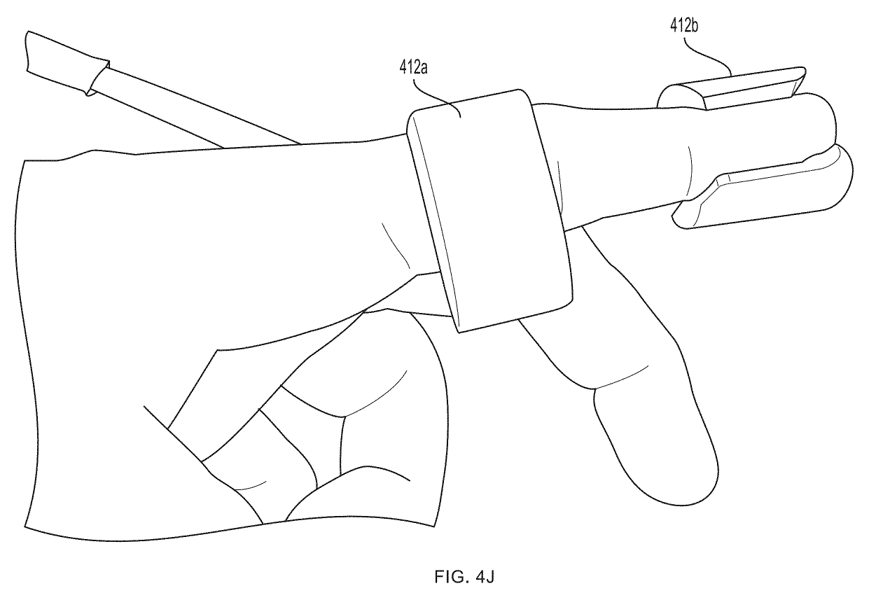

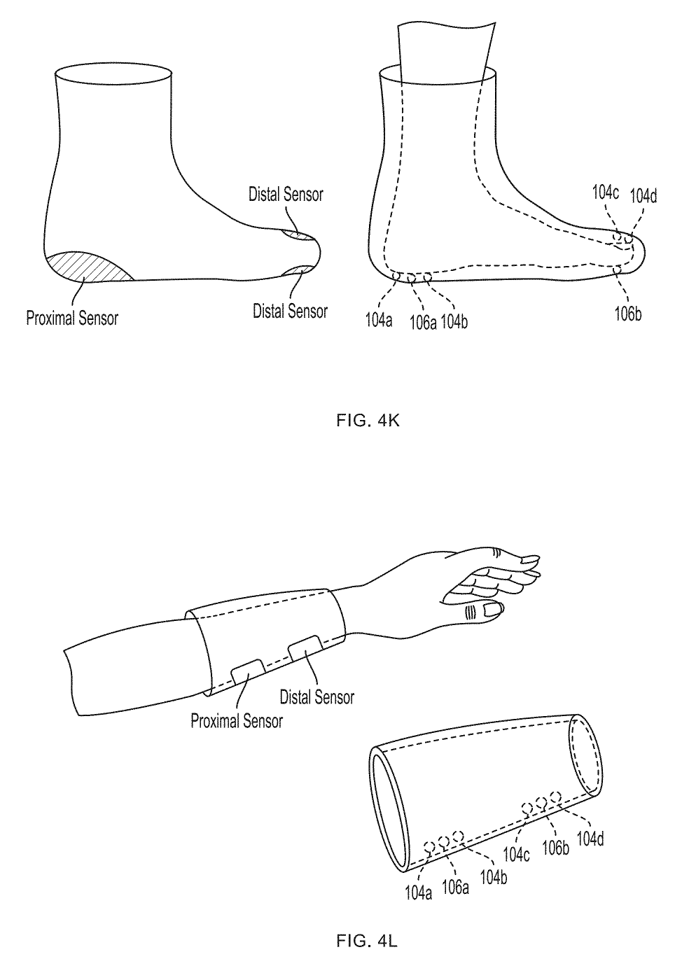

In one embodiment, the sensor 100 includes a support structure (e.g., a tether, sock, glove or sleeve) having a configuration specifically designed to register with a specific portion of the human anatomy, e.g., a finger, a hand, a forearm, etc., and the sensor bodies are arranged on the support structure in predetermined locations corresponding to the intended locations and spacing desired for the sensor(s) on the human anatomy, e.g., by mounting them on or to a substrate such as a flexible glove or flexible sleeve. The support structure thereby acts somewhat like a three-dimensional template or jig for arranging the sensors on the human anatomy in a desired spatial arrangement relative to one another. An exemplary embodiment of such a support structure is shown in FIGS. 4A-4J. FIGS. 4K-4L illustrate exemplary embodiments of support structures designed to register with specific portions of human anatomy according to an embodiment of the present invention. In this manner, the sensor's structure assists the user in using the sensor properly, as it does not require the user to follow extensive directions, anatomical knowledge or medical expertise for proper sensor placement relative to anatomical structures, but rather simplifies the process in a manner suitable for a layperson—e.g., requiring merely placing one's hand in a glove, or one's foot in a sock.

In other embodiments, the sensor may include a support structure that is more generic, and capable of registering with distinctly different parts of the human anatomy, such a spring-loaded clip or clamp.

### Control of Non-Invasive Blood Pressure Sensor

In various embodiments, each light source of one or more light sources 104 can be activated at different times such that only one light source 104 is activated at a time. For example, as depicted in FIG. 2, the resulting light received by photodetector(s) 106 can be associated with a particular light source 104 (and color) based on a time delay between activation of a particular light source 104 and later detection by the photodetector(s) 106.

Referring now to FIG. 3, a method 300 of controlling a non-invasive blood pressure sensor is provided. While specific steps in a predetermined order are illustrated in FIG. 3, in various embodiments, one or more of the steps may be excluded and/or additional steps can be added. Further, the steps may be performed in any order.

In step S302, a light source is controlled to emit a first light signal. In various embodiments, this can include controlling the light source to emit a light signal at a specific wavelength of light. In one embodiment, each of the light sources can be controlled to serially apply each light signal at a specific wavelength (e.g., blue, then green, then red, then infrared, although any order can be used). The light sources can be applied at non-overlapping periods of time. In various embodiments, the light sources can be turned on and off at such a frequency (e.g., 60 Hz or greater) that the light sources may appear to be continuously illuminated to the human eye.

In step S304, a resulting light can be detected by the one or more photodetectors. A controller can be programmed to monitor and record detected light based on the sequence of emission on step S302. For example, light can be first detected in the blue wavelength, then green, then red, then infrared. A waveform is observed wherein the peaks correspond to the pulsatile blood flow during systole and the trough is the resting phase of diastole. The difference between the peak and the trough is the measured amplitude of interest.

In step S306, the resulting light signal can be validated based on expected ranges of values (e.g., to confirm that the light sources and photodetector(s) are properly positioned). In various embodiments, validation is performed each time a measurement is performed. In other embodiments, validation is performed after the meter has been applied to a subject and once the device has been validated, validation is no longer performed. In yet other embodiments, validation is performed based upon subject-supplied commands or when the measured blood pressure levels deviate from an expected range.

In step S308, the resulting light signal can be preprocessed (e.g., by averaging over several heartbeats or other statistical techniques) to remove or minimize noise, outliers, or other variations.

Various techniques for validating and preprocessing data in the pulse oximetry field as well as hardware for implementing the same are described in John TB Moyle, Pulse Oximetry (2d ed. 2002) and can be applied prior to calculating of a blood pressure level.

In step S310, the subject's blood pressure level can be calculated as described below.

The method can then be repeated continuously or periodically to provide updated blood pressure levels. The calculation, preprocessing, validation detection, and controlling of light emission can be performed by the controller 108 of the sensor/meter.

### Calculation of Blood Pressure Level

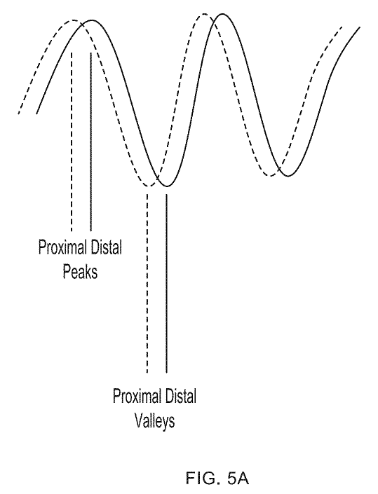

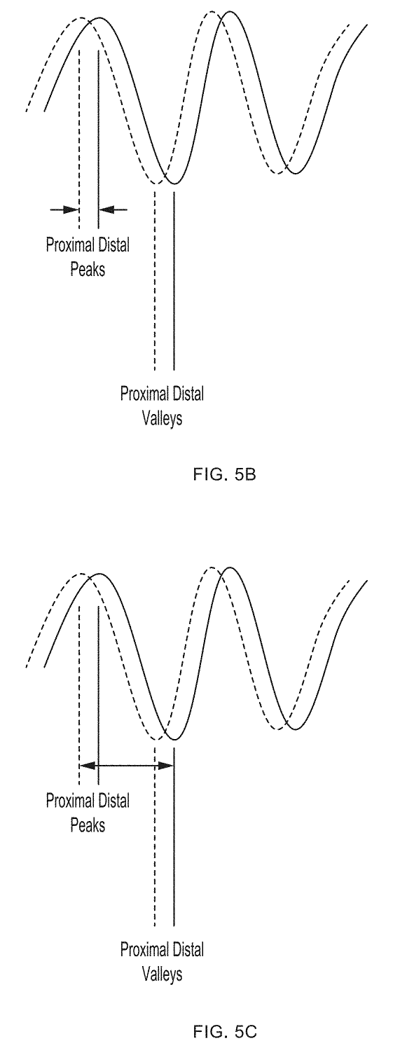

Embodiments of the invention can calculate blood pressure levels based on times between peaks and valleys as discussed below. Parameter PTT is calculated as the difference between a proximal peak calculated by an upstream light-source-and-photodetector pair and a distal peak calculated by a downstream light-source-and-photodetector pair as depicted in FIG. 5B and can be used to calculate systolic blood pressure (SBP). Parameter PTTV is calculated as the difference between a proximal peak calculated by an upstream light-source-and-photodetector pair and a distal valley calculated by a downstream light-source-and-photodetector pair as depicted in FIG. 5C and can be used to calculate diastolic blood pressure (DBP). In both parameters, the term “proximal” refers to a signal obtained from a photodetector that is upstream or closer to the heart than the “distal” signal. FIG. 5A depicts two pulsatile waveforms superimposed on the same graph. A dashed line illustrates the waveform from the proximal (or upstream) light-source-and-photodetector pair and the waveform with the solid line is from the distal (or downstream) light-source-and-photodetector pair. The offset between the two waveforms is the pulse transit time.

Parameter HR is the subject's pulse rate in beats per minute, which can be determined based on the peaks or valleys calculated by any of the photodetectors 106. For example, 60 seconds can be divided by the inter-peak (or inter-valley) time (in seconds). In another example, the number of peaks (or valleys) within 60 seconds (or other period such as 5, 10, 15, or 30 seconds can be counted).

Systolic blood pressure SBP can be calculated (e.g., by controller 108) using the exemplary Equation (1) below.

$$SBP = ( α ) [ ( β ) ( \frac{( γ ) ( 15 / PTT ( ɛ ) )}{( δ ) ( HR / 60 )} ) ] \qquad ( 1 )$$

Exemplary calibration values for Equation (1) are provided in Table 2 below.

TABLE 2
Exemplary Calibration Values

| | |
|---|---|
| α | 64.8705 |
| β | 1413.7155 |
| γ | 0.0004 |
| δ | 0.1 |
| ε | 0.00010417 |

Systolic blood pressure DBP can be calculated (e.g., by controller 108) using the exemplary Equation (2) below.

$$DBP = ( {α}^{′} ) [ ( {β}^{′} ) ( \frac{( {γ}^{′} ) ( 15 / PTTV ( {ɛ}^{′} )}{( {δ}^{′} ) ( HR / 60 )} ) ] \qquad ( 2 )$$

Exemplary calibration values for Equation (2) are provided in Table 3 below.

TABLE 3
Exemplary Calibration Values

| | |
|---|---|
| α′ | 64.7501 |
| β′ | 1413.7155 |
| γ′ | 0.0004 |
| δ′ | 0.1 |
| ε′ | 0.00010417 |

The calculated blood pressure and/or pulse values can be displayed, communicated, and/or stored by controller 108.

Although exemplary calibration values are provided for Equations (1) and (2), a person of ordinary skill in the art will appreciate that these calibration values may vary for a particular implementation (e.g., using light sources 104 of varying spectra and/or intensity, photodetectors 106 of varying spectra and/or sensitivity, contemplated placement of sensor 100, and the like). Particular calibration values for a given embodiment can be determined by obtaining amplitude values for a plurality of wavelengths and blood pressure levels obtained by other methods (e.g., using a sphygmomanometer) for a test population of subjects. Various fitting algorithms can be used to optimize the calibration values to minimize errors in prediction. Exemplary algorithms are described in treatises such as Rudolf J. Freund et al., Regression Analysis (2d ed. 2006); P. G. Guest, Numerical Methods of Curve Fitting (1961); and Harvey Motulsky & Arthur Christopoulos, Fitting Models to Biological Data Using Linear and Nonlinear Regression (2003).

Additionally, the calibration values can be fit to a particular subject using the same techniques. Even without fitting, the device can still track trends for feedback to the subject.

Communication with Other Devices

Embodiments of the non-invasive blood pressure sensor 100 can be designed for repeated use or single use and can use one or more communication links for communicating with a controller 108 as will be further described herein. For example, the non-invasive blood pressure sensor 100 can implement one or more wired or wireless communication protocols.

In one embodiment, the non-invasive blood pressure sensor 100 can include the appropriate hardware and/or software to implement one or more of the following communication protocols: Universal Serial Bus (USB), USB 2.0, IEEE 1394, Peripheral Component Interconnect (PCI), Ethernet, Gigabit Ethernet, and the like. The USB and USB 2.0 standards are described in publications such as Andrew S. Tanenbaum, Structured Computer Organization Section § 3.6.4 (5th ed. 2006); and Andrew S. Tanenbaum, Modern Operating Systems 32 (2d ed. 2001). The IEEE 1394 standard is described in Andrew S. Tanenbaum, Modern Operating Systems 32 (2d ed. 2001). The PCI standard is described in Andrew S. Tanenbaum, Modern Operating Systems 31 (2d ed. 2001); Andrew S. Tanenbaum, Structured Computer Organization 91, 183-89 (4th ed. 1999). The Ethernet and Gigabit Ethernet standards are discussed in Andrew S. Tanenbaum, Computer Networks 17, 65-68, 271-92 (4th ed. 2003).

In other embodiments, the non-invasive blood pressure sensor 100 can include appropriate hardware and/or software to implement one or more of the following communication protocols: BLUETOOTH®, IEEE 802.11, IEEE 802.15.4, and the like. The BLUETOOTH® standard is discussed in Andrew S. Tanenbaum, Computer Networks 21, 310-17 (4th ed. 2003). The IEEE 802.11 standard is discussed in Andrew S. Tanenbaum, Computer Networks 292-302 (4th ed. 2003). The IEEE 802.15.4 standard is described in Yu-Kai Huang & Ai-Chan Pang, “A Comprehensive Study of Low-Power Operation in IEEE 802.15.4” in MSWiM'07 405-08 (2007).

### Controller

The non-invasive blood pressure sensors can be sold as stand-alone peripheral devices, or a non-invasive blood pressure sensor 100 can be sold as an integrated meter device including sensors 102 and/or a controller 108 and/or a display device 110.

In one embodiment, the non-invasive blood pressure sensor 100 includes a controller 108 configured to obtain resulting signals from the one or more photodetectors 106 of the sensor 102. Controller 108 can be further configured to provide instructions to each light source 104 to emit light and to each photodetector 106 to measure resulting light intensities.

Controller 108 can be disposed on sensor body 102 or on a substrate separate from sensor body 102. In one embodiment, the controller 108 filters, processes and/or converts the resulting signal or signals to determine a blood pressure value for a subject.

Controller 108 can either be a fixed unit that handles all aspects of control and measurement and outputs a blood pressure level (and potentially other measurements), e.g., through a display or communication with another device, or can rely on an external device (e.g., a smartphone or a computer) including software and/or hardware including instructions for controlling the operation of light source(s) 104 and photodetectors 106 and calculating blood pressure levels based on the received values.

Controller 108 can be an electronic device programmed to control the operation of the system to achieve a desired result. The controller 108 can be programmed to autonomously determine a blood pressure level in a subject based upon emission and detection of light.

Controller 108 can be a computing device such as a general purpose computer (e.g., a personal computer (“PC”), laptop, desktop), workstation, mainframe computer system, a patient telemetry device, a smartphone (e.g., a device sold under the IPHONE® trademark by Apple, Inc. of Cupertino, Calif., the WINDOWS® trademark by Microsoft Corporation of Redmond Wash., the ANDROID™ trademark by Google Inc. of Mountain View, Calif., and the like), a tablet (e.g., devices sold under the IPAD® trademark from Apple Inc. of Cupertino, Calif. and the KINDLE® trademark from Amazon Technologies, LLC of Reno, Nev. and devices that utilize WINDOWS® operating systems available from Microsoft Corporation of Redmond, Wash. or ANDROID® operating systems available from Google Inc. of Mountain View, Calif.), a video game console (e.g., the WII U® console available from Nintendo of America Inc. of Redmond, Wash.; the SONY® PLAYSTATION™ console available from Kabushiki Kaisha Sony Corporation of Tokyo, Japan; the MICROSOFT® XBOX™ console available from Microsoft Corporation of Redmond, Wash.), smart speaker devices (e.g., devices sold under the AMAZON ECHO™ trademark from Amazon Technologies, LLC of Reno, Nev., the GOOGLE HOME™ trademark by Google Inc. of Mountain View, Calif., and the CASTLEHUB® trademark by CastleOS Software, LLC of Johnston, R.I.), medical devices (e.g., insulin pumps, hospital monitoring systems, intravenous (IV) pumps), electronic medical record (EMR) systems, electronic health record (EHR) systems, and the like.

Controller 108 can include a processor device (or central processing unit “CPU”), a memory device, a storage device, a user interface, a system bus, and/or a communication interface.

A processor can be any type of processing device for carrying out instructions, processing data, and so forth.

A memory device can be any type of memory device including any one or more of random access memory (“RAM”), read-only memory (“ROM”), Flash memory, Electrically Erasable Programmable Read Only Memory (“EEPROM”), and so forth.

A storage device can be any data storage device for reading/writing from/to any removable and/or integrated optical, magnetic, and/or optical-magneto storage medium, and the like (e.g., a hard disk, a compact disc-read-only memory “CD-ROM”, CD-ReWritable “CD-RW”, Digital Versatile Disc-ROM “DVD-ROM”, DVD-RW, and so forth). The storage device can also include a controller/interface for connecting to a system bus. Thus, the memory device and the storage device can be suitable for storing data as well as instructions for programmed processes for execution on a processor.

The user interface can include a touch screen, control panel, keyboard, keypad, display, voice recognition and control unit, or any other type of interface, which can be connected to a system bus through a corresponding input/output device interface/adapter.

The communication interface can be adapted and configured to communicate with any type of external device. The communication interface can further be adapted and configured to communicate with any system or network, such as one or more computing devices on a local area network (“LAN”), wide area network (“WAN”), the Internet, and so forth. The communication interface can be connected directly to a system bus or can be connected through a suitable interface.

The controller 108 can, thus, provide for executing processes, by itself and/or in cooperation with one or more additional devices, that can include algorithms for controlling various components of the light sources and photodetector(s) in accordance with the present invention. Controller 108 can be programmed or instructed to perform these processes according to any communication protocol and/or programming language on any platform. Thus, the processes can be embodied in data as well as instructions stored in a memory device and/or storage device or received at a user interface and/or communication interface for execution on a processor.

The controller 108 can control the operation of the system components in a variety of ways. For example, controller 108 can modulate the level of electricity provided to a component. Alternatively, the controller 108 can transmit instructions and/or parameters a system component for implementation by the system component.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like), non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### Working Example

Referring now to the sensor 400 shown in FIGS. 4A-4J, a first pair of light sources 404a, 404b (e.g., blue light source 404a and green light source 404b) and a first photodetector 406a is located within a first sensor body 412a at the base (e.g., over a proximal phalanx) of a finger while a second pair of light sources 404c, 404d (e.g., red light source 404c and infrared light source 404d) and a second photodetector 406b is located within a second sensor body 412b positioned over a tip of the same finger. As further described in U.S. Provisional Patent Application Ser. No. 62/417,226, filed Nov. 3, 2016, under Attorney Docket No. 368114.00005(P2), distribution of light sources 404a, 404b, 404c, 404d and photodetectors 406a, 406b along a limb (e.g., a finger) facilitates measurement of blood pressure using pulse transit time, as determined by the controller 408. (An additional optional pulse oximetry sensor 414 is also depicted in FIGS. 4A and 4B, but is not essential to the invention described herein.)

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

### APPENDIX

TABLE 4
Exemplary Components

| Component | Source | Product No. |
|---|---|---|
| Blue LED | Kingbright | APT1608LVBC/D |
| Green LED | Kingbright | APT1608LZGCK |
| Red LED | Lite-On Electronics, Inc. | LTST-C171CKT |
| Infrared LED | SunLED | XZTNI54W |

1. A non-invasive blood pressure sensor comprising:
one or more sensor bodies configured to mate with a tissue surface;
a first light-source-and-photodetector pair disposed on one of the one or more sensor bodies in a pre-determined spatial relationship over a proximal anatomical location on a subject; and
a second light-source-and-photodetector pair disposed on one of the one or more sensor bodies in a pre-determined spatial relationship over a distal anatomical location on the subject.

2. The non-invasive blood pressure sensor of claim 1, wherein the first light-source-and-photodetector pair and the second light-source-and-photodetector pair are disposed on the one or more sensor bodies in a pre-determined spatial relationship with respect to each other.

3-5. (canceled)

6. The non-invasive blood pressure sensor of claim 1, wherein the support structure is a flexible and selected from the group consisting of: a strap, a glove, a cuff, and a sleeve, each of which is configured to register with a corresponding portion of human anatomy in a predetermined fashion, to support the light-source-and-photodetector pairs in a pre-determined spatial relationship with respect to the corresponding portion of human anatomy.

7. (canceled)

8. The non-invasive blood pressure sensor of claim 1, wherein the pre-determined spatial relationships are selected to facilitate capturing light emanating from the tissue surface after one or more path selected from the group consisting of: transmission, reflection, and transflection.

9. (canceled)

10. The non-invasive blood pressure sensor of claim 1, wherein the light-source-and-photodetector pairs each comprise a light source and a photodetector, and wherein the one or more sensor bodies are configured to hold the light sources and the photodetectors such that when the one or more sensor bodies are pressed against the tissue surface, the photodetectors are shielded from ambient light such that the photodetectors only measure light emerging from the tissue surface after emission by the light sources.

11. (canceled)

12. The non-invasive blood pressure sensor of claim 1, wherein the light-source-and-photodetector pairs each comprise a light source and a photodetector, and wherein the pre-determined spatial relationship is selected to cause the photodetectors to lie adjacent the light sources when the one or more sensor bodies mate with the tissue surface and to receive light emitted by the light sources after reflection or transflection.

13. The non-invasive blood pressure sensor of claim 1, wherein the light-source-and-photodetector pairs each comprise a light source and a photodetector, and wherein the pre-determined spatial relationship is selected to cause the photodetectors to lie on an opposite tissue surface from the light sources when the one or more sensor bodies mate with the tissue surface and to receive light emitted by the light sources after transmission.

14. The non-invasive blood pressure sensor of claim 1, wherein the light-source-and-photodetector pairs each comprise a light source and a photodetector, and wherein the light sources emit light having a color selected from the group consisting of: ultraviolet, violet, blue, green, yellow, orange, red, near infrared, and infrared.

15. The non-invasive blood pressure sensor of claim 1, wherein the light-source-and-photodetector pairs each comprise a light source and a photodetector, and further comprising:
a controller programmed to:
receive one or more signals from the photodetectors; and
calculate blood pressure values as function of at least the one or more signals received from the photodetectors after emission by the light sources.

16. The non-invasive blood pressure sensor of claim 15, wherein the controller is further programmed to control selective actuation of the light sources.

17. (canceled)

18. The non-invasive blood pressure sensor of claim 15, wherein the controller is further programmed to:
identify a plurality of peaks and valleys over a time series of data obtained from the photodetectors; and
calculate the subject's blood pressure based on differences in time between:
a proximal peak detected by the first light-source-and-photodetector pair and a distal peak detected by the second light-source-and-photodetector pair; and
a proximal peak detected by the first light-source-and-photodetector pair and a distal valley detected by the second light-source-and-photodetector pair.

19. The non-invasive blood pressure sensor of claim 18, wherein the controller is further programmed to calculate the blood pressure using the equations:
$$SBP = ( α ) [ ( β ) ( \frac{( γ ( 15 / PTT ( ɛ )}{( δ ) ( HR / 60 )} ) ] and \\ DBP = ( {α}^{′} ) [ ( {β}^{′} ) \frac{( {γ}^{′} ) ( 15 / PTTV ( {ɛ}^{′} )}{( {δ}^{′} ) ( HR / 60 )} ) ] , wherein \text{:}$$
PTT is a difference between the proximal peak detected by the first light-source-and-photodetector pair and the distal peak detected by the second light-source-and-photodetector pair in seconds;
PTTV is a difference between the proximal peak detected by the first light-source-and-photodetector pair and the distal valley detected by the second light-source-and-photodetector pair in seconds;
HR is the subject's pulse rate in beats per minute; and
α, β, γ, δ, ε, α′, β′, γ′, δ′, and ε′ are calibration constants.

20. (canceled)

21. (canceled)

22. The non-invasive blood pressure sensor of claim 1, wherein the first light-source-and-photodetector pair and the second light-source-and-photodetector pair are both mounted on a glove dimensioned and configured to receive a human hand.

23. The non-invasive blood pressure sensor of claim 1, wherein the first light-source-and-photodetector pair and the second light-source-and-photodetector pair are both mounted on a sleeve dimensioned and configured to receive a human limb.

24. The non-invasive blood pressure sensor of claim 1, further comprising:
a stethoscope, wherein the first light-source-and-photodetector pair is mounted in the stethoscope; and
a watch or wristband, wherein the second light-source-and-photodetector pair is mounted in the watch or wristband.

25. (canceled)

26. A non-invasive blood pressure sensor comprising:
a proximal optical arrangement adapted and configured for mounting in a proximal anatomical location on a subject, the proximal optical arrangement comprising:
one or more first light sources; and
one or more first photodetectors positioned to measure light from one or more of the one or more first light sources after transmission, reflection, or transflection from the subject's skin; and
a distal optical arrangement adapted and configured for mounting in a distal anatomical location on the subject, the distal optical arrangement comprising:
one or more second light sources; and
one or more second photodetectors positioned to measure light from one or more of the one or more second light sources after transmission, reflection, or transflection from the subject's skin.

27. The non-invasive blood pressure sensor of claim 26, further comprising:
a controller programmed to:
identify a plurality of peaks and valleys over a time series of data obtained from the one or more first photodetectors and the one or more second photodetectors;
calculate the subject's blood pressure based on differences in time between:
a proximal peak detected by the proximal optical arrangement and a distal peak detected by the distal optical arrangement; and
a proximal peak detected by the proximal optical arrangement and a distal valley detected by the distal optical arrangement.

28. The non-invasive blood pressure sensor of claim 27, wherein the controller is further programmed to calculate the blood pressure using the equations:
$$SBP = ( α ) [ ( β ) ( \frac{( γ ( 15 / PTT ( ɛ )}{( δ ) ( HR / 60 )} ) ] and \\ DBP = ( {α}^{′} ) [ ( {β}^{′} ) \frac{( {γ}^{′} ) ( 15 / PTTV ( {ɛ}^{′} )}{( {δ}^{′} ) ( HR / 60 )} ) ] , wherein \text{:}$$
PTT is a difference between the proximal peak detected by the proximal optical arrangement and the distal peak detected by the distal optical arrangement in seconds;
PTTV is a difference between the proximal peak detected by the proximal optical arrangement and the distal valley detected by the distal optical arrangement in seconds;
HR is the subject's pulse rate in beats per minute; and
α, β, γ, δ, ε, α′, β′, γ′, δ′, and ε′ are calibration constants.

29. The non-invasive blood pressure sensor of claim 28, wherein:
α is about 64.8705;
β is about 1413.7155;
γ is about 0.0004;
δ is about 0.1;
ε is about 0.00010417;
α′ is about 64.7501;
β′ is about 1413.7155;
γ′ is about 0.0004;
δ′ is about 0.1; and
ε′ is about 0.00010417.

30. The non-invasive blood pressure sensor of claim 27, wherein the proximal optical arrangement, the distal optical arrangement, and the controller are housed in a unitary assembly.

31. (canceled)

32. (canceled)

33. The non-invasive blood pressure sensor of claim 26, wherein the proximal optical arrangement and the distal optical arrangement are both mounted on a glove dimensioned and configured to receive a human hand.

34. The non-invasive blood pressure sensor of claim 26, wherein the proximal optical arrangement and the distal optical arrangement are both mounted on a sleeve dimensioned and configured to receive a human limb.

35. (canceled)

36. (canceled)

37. A non-invasive blood pressure sensor comprising:
one or more sensor bodies configured to mate with a tissue surface;
a first light-source-and-photodetector pair disposed on one of the one or more sensor bodies in a pre-determined spatial relationship over a proximal anatomical location on a subject;
a second light-source-and-photodetector pair disposed on one of the one or more sensor bodies in a pre-determined spatial relationship over a distal anatomical location on the subject, wherein each of said light-source-and-photodetector pairs comprises a respective light source and a respective photodetector, and wherein the pre-determined spatial relationships are selected to facilitate capturing light emanating from the tissue surface after one or more path selected from the group consisting of: transmission, reflection, and transflection; and
a controller programmed to:
receive one or more signals from the photodetectors; and
calculate blood pressure values as function of at least the one or more signals received from the photodetectors after emission by the light sources.
